(19) United States
(12) Patent Application Publication
(10) Pub. No.: US 2002/0051990 A1
(43) Pub. Date: May 2, 2002

(54) Novel gene targets and ligands that bind thereto for treatment and diagnosis of ovarian carcinomas

(76) Inventors: Eric Ople, La Jolla, CA (US); Karen McLachlan, Solana Beach, CA (US); Cheryl J. Heard, Encinitas, CA (US)

Correspondence Address: Pillsbury Winthrop LLP, Intellectual Property Group, East Tower, Ninth Floor, 1100 New York Avenue, N.W., Washington, DC, 20005-3918, US

(21) Appl. No.: 09/877,065

(22) Filed: Jun. 11, 2001

Related U.S. Application Data
(60) Provisional application No. 60/210,451, filed on Jun. 9, 2000.

(51) Int. Cl. C12Q 1/68; C07H 21/04

(52) U.S. Cl. 435/6; 536/23.2; 536/24.3

(57) ABSTRACT

Two genes that correlate to human ovarian cancers are provided. These genes and the corresponding antigens are useful diagnostic and therapeutic targets.

### CROSS REFERENCE TO RELATED APPLICATION

This application claims priority from U.S. Ser. No. 60/210,451, filed Jun. 9, 2000, and is incorporated herein by reference in its entirety.

### FIELD OF THE INVENTION

The present invention relates to two novel gene targets for treatment and diagnosis of cancers, especially ovarian cancer, and the use thereof to express the corresponding antigen, and to produce ligands that specifically bind such antigen, e.g. monoclonal antibodies and small molecules.

### BACKGROUND OF THE INVENTION

Ovarian cancer is a disorder that affects thousands of women annually. Unfortunately, it is a cancer that is usually not detected until the disease has progressed to a fairly advanced stage. Consequently, a large percentage of women diagnosed with the disease do not survive.

Currently, there do not exist may effective therapies for ovarian cancer. Generally, treatment of ovarian cancer comprises surgical removal of the ovaries and any other tissues to which the cancer may have spread, followed by chemotherapy or radiation or a combination thereof. For example, the use of Taxol and certain growth factors or hormones, e.g., progestin and EGF in treatment of ovarian cancer have been reported.

In the past ten to fifteen years, various gene targets have been identified, the presence of which correlates to the presence of particular types of ovarian cancers.

For example, it has been reported that specific BRCA2 gene alleles correlate to persons having a predisposition to develop breast and ovarian cancer. (See U.S. Pat. No. 6,045,997, issued Apr. 4, 2000, to Futreol et al. and assigned to Duke University and Cancer Research Campain Technology Limited.)

Also, it has been reported that the presence of specific erbB-2 genes, and ligands thereto correlate to a predisposition for developing breast and ovarian cancer, and that these genes and ligands are useful targets for treatment and diagnosis. (See U.S. Pat. No. 6,040,290, issued Mar. 27, 2000, to Lippman et al., assigned to Georgetown University, which teaches ligand growth gp30 that binds to erbB-2 receptor protein; U.S. Pat. No. 6,037,134, issued Mar. 17, 2000, to Margolis and U.S. Pat. No. 6,001,583 issued Dec. 14, 1999, assigned to New York University, Medical Center, which teach HER2/GRB-7 complexes, the presence of which correlates to certain breast and ovarian cancers; and U.S. Pat. Nos. 5,772,997, 5,770,195 issued to Hudziak and assigned to Genentech, issued respectively on Jun. 30, 1998 and Jun. 23, 1998, as well as U.S. Pat. Nos. 5,725,856 and 5,729,954, issued respectively on Mar. 10, 1998 and Feb. 24, 1998, and assigned to Genentech, which teach monoclonal antibodies to HER2 receptor.

Further, the use of antisense oligonucleotides to treat cancers including breast and ovarian carcinomas has been reported, e.g., U.S. Pat. No. 6,007,997, issued Dec. 28, 1999, to Sivaraman et al. and assigned to the Research Foundation of SUNY, which discloses the use of antibodies oligos complementary to ERR-1 or ERR-2 to treat ovarian and breast cancer. Also, U.S. Pat. No. 5,968,748 to Bennett et al., assigned to ISIS Pharmaceutical and Pennsylvania State Research Foundation, discloses the use of HER2 anti-sense oligos to treat breast and ovarian cancers.

Still further, it has been reported that TAT1 (tumor associated trypsin inhibitor) is a marker of ovarian cancer (Medl et al., Br. J. Cancer 71:1051-1054 (1995)). Also, the use of EGFR as a target for advanced ovarian cancer has been reported (Scambia et al., J. Clin Oncol, 10:529-535 (1992).

Moreover, BRCA-1 protein kinase has been reported to be a useful diagnostic and treatment target for ovarian cancer. (See U.S. Pat. No. 5,972,675 issued Oct. 26, 1999 to Backmann et al., assigned to Eli Lilly and Company; U.S. Pat. No. 5,891,857 issued Apr. 6, 1999 to Holt et al., and jointly assigned to Vanderbilt University and the University of Washington.)

Further, the new gene has been reported to be a useful target for treating cancers affecting the female genital tract (See U.S. Pat. No. 5,814,315 issued Sep. 29, 1999 to Hing, et al. and assigned to University of Texas).

Also, the detection of breast or ovarian cancer based on the detection of mutated forms of the progesterone receptor gene has been reported (U.S. Pat. No. 5,683,885, issued Nov. 4, 1997, to Kieback, and U.S. Pat. No. 5,645,995 issued Jul. 8, 1997, both of which are assigned to Baylor College of Medicine.)

Further, the use of the glycoprotein Mullerian Inhibiting Substance (MIS) as a target for treating certain tumors, including ovarian tumors, has been reported (See U.S. Pat. No. 5,661,126 issued Aug. 26, 1997 to Donahoe et al., and U.S. Pat. No. 5,547,856 issued Aug. 20, 1996, and assigned to General Hospital Corporation). Additionally, it has been reported by several groups recently that CA125 is a suitable antigen to target for ovarian cancer therapies.

However, notwithstanding what has been reported, there exists a significant need for the identification of novel gene targets for the treatment and diagnosis of ovarian cancer, especially given the huge human toll caused by this disease annually.

### OBJECTS OF THE INVENTION

It is an object of the invention to identify novel gene targets for treatment and to diagnosis of ovarian cancer.

It is a specific object of the invention to develop novel therapies for treatment of ovarian cancer involving the administration of anti-sense oligonucleotides corresponding to novel gene targets that are expressed by certain ovarian cancers.

It is another specific object of the invention to provide the antigens expressed by genes that are exposed by certain ovarian cancer.

It is another specific object of the invention to produce ligands that bind antigens expressed by certain ovarian cancers, especially monoclonal antibodies.

It is another specific object of the invention to provide novel therapeutic regimens for the treatment of ovarian cancer that involve the administration of antigens expressed by certain ovarian cancers, alone or in combination with adjuvants that elicit an antigen-specific cytotoxic T-cell lymphocyte response against cancer cells that express such antigen.

It is another object of the invention to provide novel therapeutic regimens for the treatment of ovarian cancer that involve the administration of ligands, especially monoclonal antibodies that specifically bind novel antigens that are expressed by certain ovarian cancers.

It is another object of the invention to provide a novel method for diagnosis of ovarian cancer by using ligands, e.g., monoclonal antibodies, that specifically bind to antigens that are expressed by certain ovarian cancers, in order to detect whether a subject has or is at increased risk of developing ovarian cancer.

It is another object of the invention to provide a novel method of detecting persons having, or at increased risk of developing ovarian cancer by use of labelled DNAs that hybridize to novel gene targets expressed by certain ovarian cancers.

It is yet another object of the invention to provide diagnostic test kits for the detection of persons having or at increased risk of developing ovarian cancer that comprise a ligand, e.g., monoclonal antibody that specifically binds to an antigen expressed by certain ovarian cancers, and a detectable label, e.g. a radiolabel or fluorophore.

It is another object of the invention to provide diagnostic kits for detection of persons having or at risk of developing ovarian cancer that comprise DNA primers or probes specific for novel gene targets expressed by ovarian cancers, and a detectable label, e.g. radiolabel or fluorophore.

### BRIEF DESCRIPTION OF THE FIGURES

FIG. 1(a) contains novel cDNA sequence identified by the present inventor which are expressed by human ovarian tumors which were identified by RDA screening. FIG. 1(b) contains the sequence of an EST accessible from the NIH under AA133536 and available from the ATCC as clone # 947812.

FIG. 2(a) contains the results of a experiment measuring the expression of gene A having the sequence shown in FIG. 1(a) in a multiple tissue northern blot using the sequence in FIG. 1(a) as a probe.

FIG. 2(b) contains result of an experiment that evaluated the expression of the gene contained in FIG. 1(a) by PCR in different normal tissues.

FIG. 3(a) contains results of a PCR experiment that measured expression of the gene contained in FIG. 1(a) in cDNA libraries obtained from different human tumors.

FIG. 3(b) contains results of a PCR experiment that measured expression of the gene contained in FIG. 1(a) in cDNA samples prepared from different ovarian tumors.

FIG. 4(a) contains the partial sequence of a second gene (gene B named “OREO” (Ople¹ RDA of Epithelial Tissue vs. Ovary Tumor) identified by the present inventors by RDA screening of human ovarian tumors.

FIG. 4(b) contains a partial sequence of the same second gene (gene B) identified by the present inventors by RDA screening of human ovarian tumors.

FIG. 4(c) contains complete sequence of an EST clone designated AI799522 in Genbank and available from ATCC unit catalog #3413715 which contains the sequence contained in FIG. 4(a).

FIG. 4(d) contains the results of a PCR experiment wherein ovarian tumor cDNA was used as a template in a PCR reaction with the primers that are underlined in sequence in FIGS. 4(a) and 4(b). The dash in the Figure indicates the location of the 507/516 bp DNA size marker.

FIG. 4(e) contains the sequence of the PCR product obtained in the PCR reaction, the results of which are contained in FIG. 4(d). The primers used are underlined in this panel as well as in FIGS. 4(a) and 4(b). The sequence is identical to nucleotides 72023 to 72594 of Genbank entry AL080312.

FIG. 5(a) contains results of a multiple tissue northern blots experiment measuring expression of second gene (B) (OREO), using sequence in FIG. 4(b) as a probe.

FIG. 5(b) contains the results of a PCR experiment that measured gene expression of the sequence contained in FIG. 4(b) in a panel of cDNAs prepared from normal tissue.

FIG. 6(a) contains the results of a PCR experiment that measured the expression of gene B in a panel of human tumors using the primer pair identified in FIG. 5.

FIG. 6(b) contains results of PCR experiment that measured expression of gene B in a panel of human ovarian tumor from cDNA samples prepared using the primer identified in FIG. 5.

FIG. 7(a) contains the complete nucleotide sequence of the Oreo gene.

FIG. 7(b) contains the predicted amino acid sequence of the open reading frame of the Oreo gene.

FIG. 8(a) contains the arrangement of the Oreo gene in the human genome.

FIG. 8(b) depicts the exon arrangement of the protein domains of the Oreo gene.

FIG. 8(c) is a schematic showing the predicted topology of Oreo in the plasma membrane.

FIG. 9 shows Oreo expression in ovarian tumor and normal cell lines as measured by Northern blot analysis.

FIG. 10 contains the results of indirect immunofluoresence experiments showing the localization of intact Oreo and Oreo-exo (predicted transmembrane domain of OREO) on the cell surface or intracellularly on transfected COS-7 cells.

FIG. 11 shows plasmid maps of the mammalian expression vector INPEP4+Leader containing (a) Intact Oreo—the entire coding sequence of the Oreo gene, and (b) Oreo-exo—the extracellular domain of Oreo.

FIG. 12 shows the plasmid map of the mammalian expression vector N5L-GFP containing the Oreo-exo, the extracellular domain of Oreo.

### DETAILED DESCRIPTION OF THE INVENTION

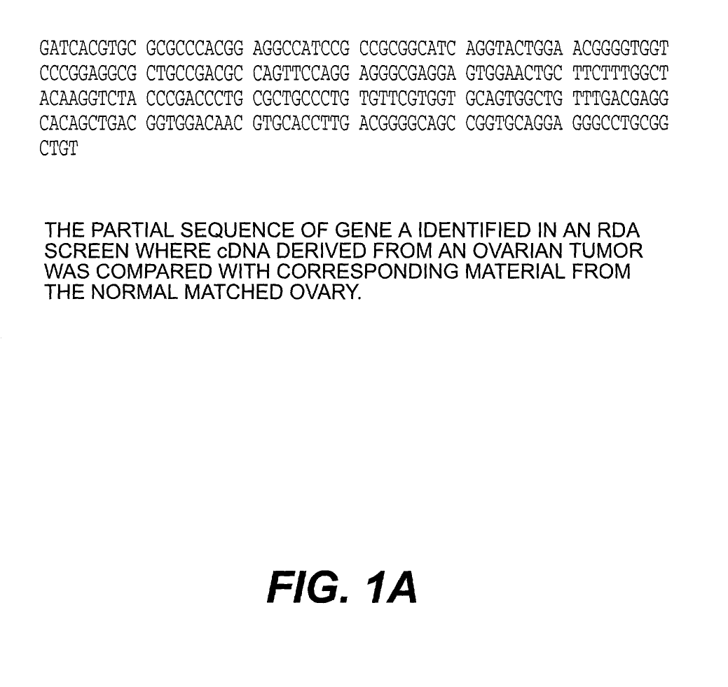

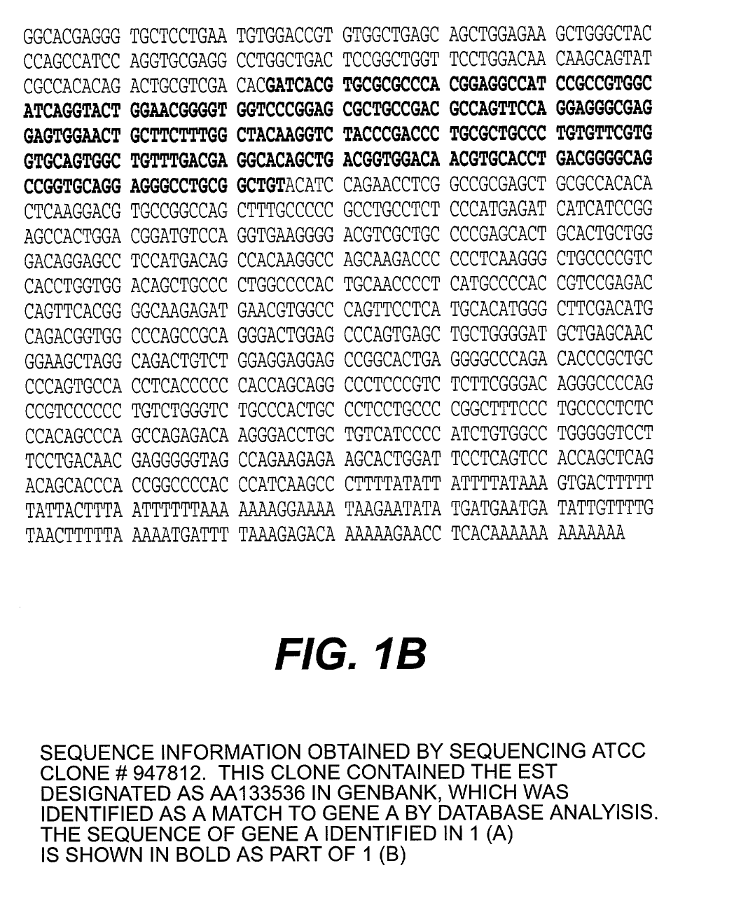

The present invention provides two novel gene targets that were identified by RDA screening, which sequences are contained in FIGS. 1(a), 4(a), 4(b) and 7(a), that are selectively expressed by certain human ovarian tumors relative to normal human ovarian tissues or other normal epithelial tissues.

As shown in the examples infra, the first gene (gene A) was identified in an RDA screen where cDNA derived from an ovarian tumor was compared with a corresponding cDNA material obtained from a matched normal ovary. FIG. 1(a) depicts the sequence identified by the RDA screen, which was found to maintain identically an EST in the dbEST database, having an EST accession number of AA133536, available through the ATCC as clone #947812.

As discussed in the examples which follow, the A gene is expressed only in normal placenta and testis but not in other normal tissues tested (colon, ovary, peripheral blood leukocytes, prostate, small intestine, thymus). Also, this gene was expressed in tissue of five ovarian tumor samples tested. Based on this expression pattern, this gene should be a useful target for ovarian cancer treatment and/or detection.

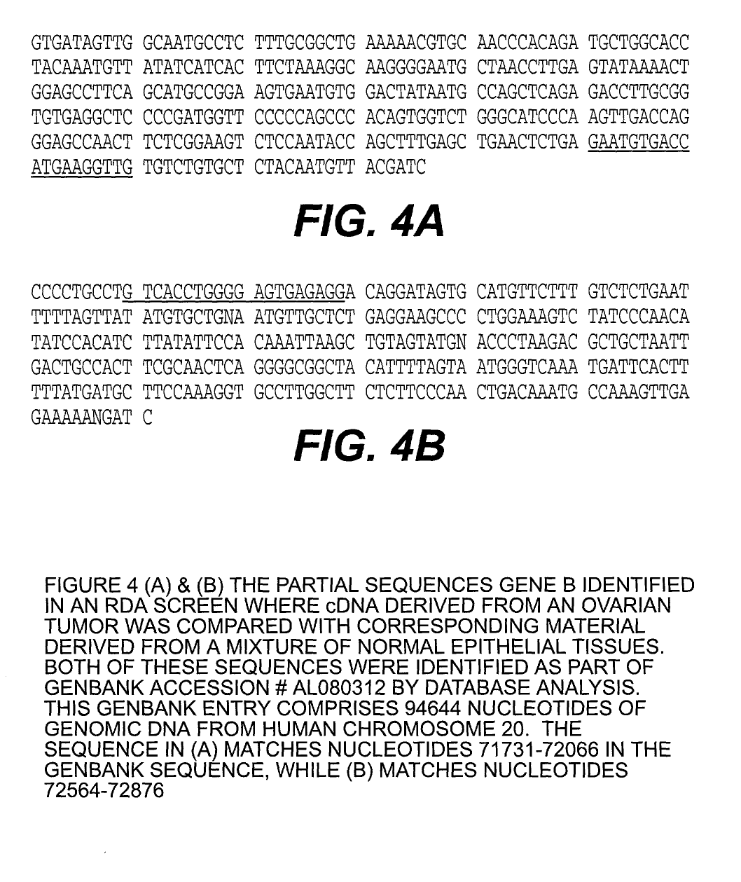

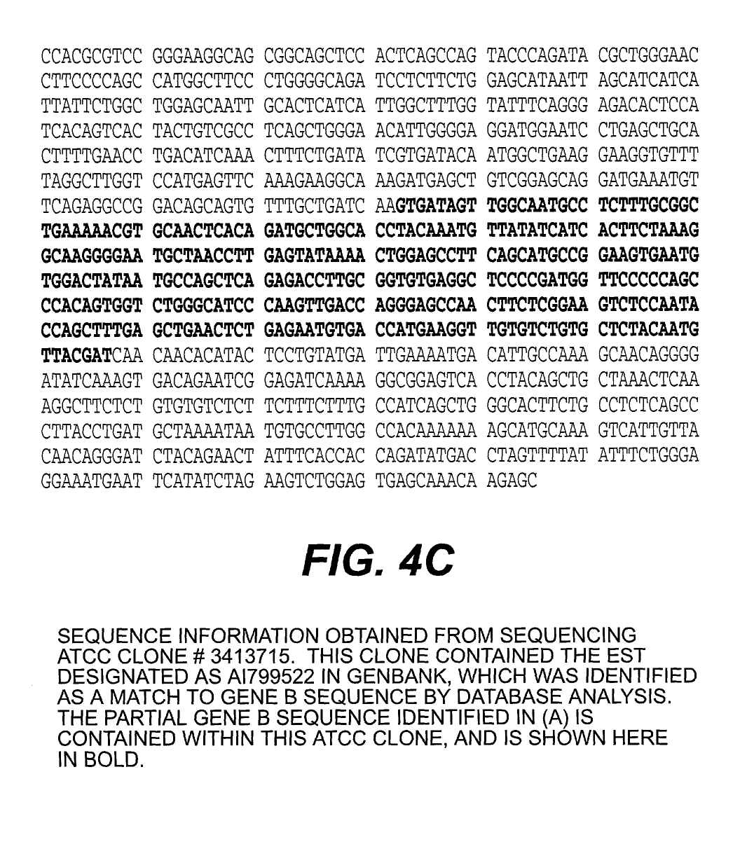

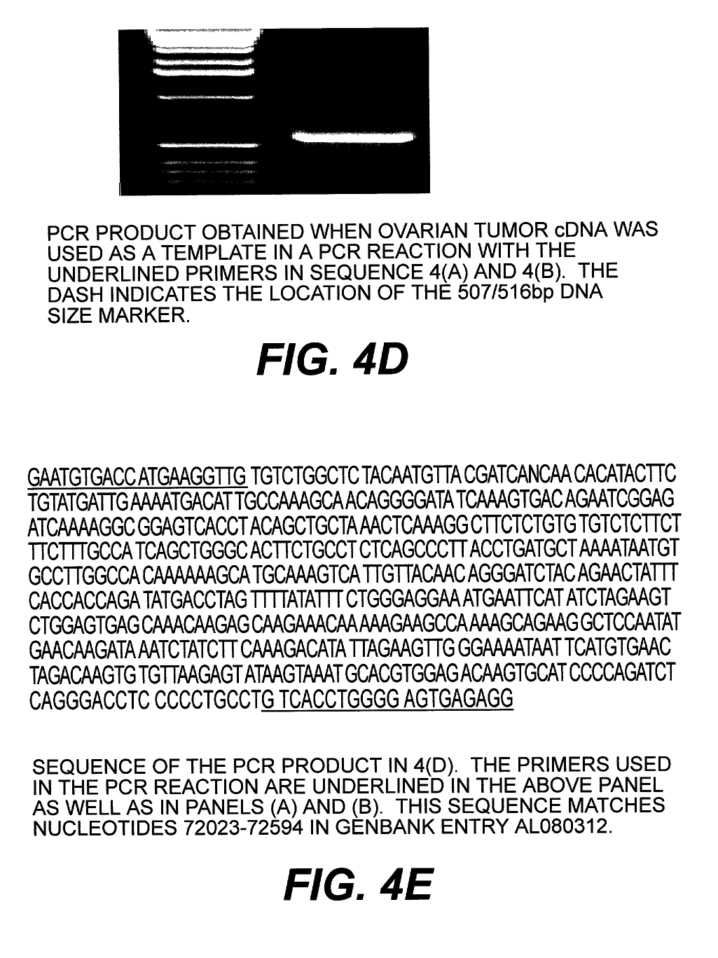

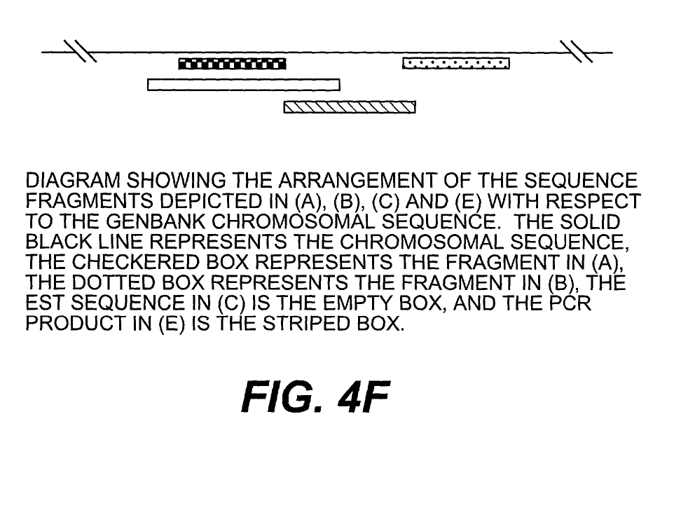

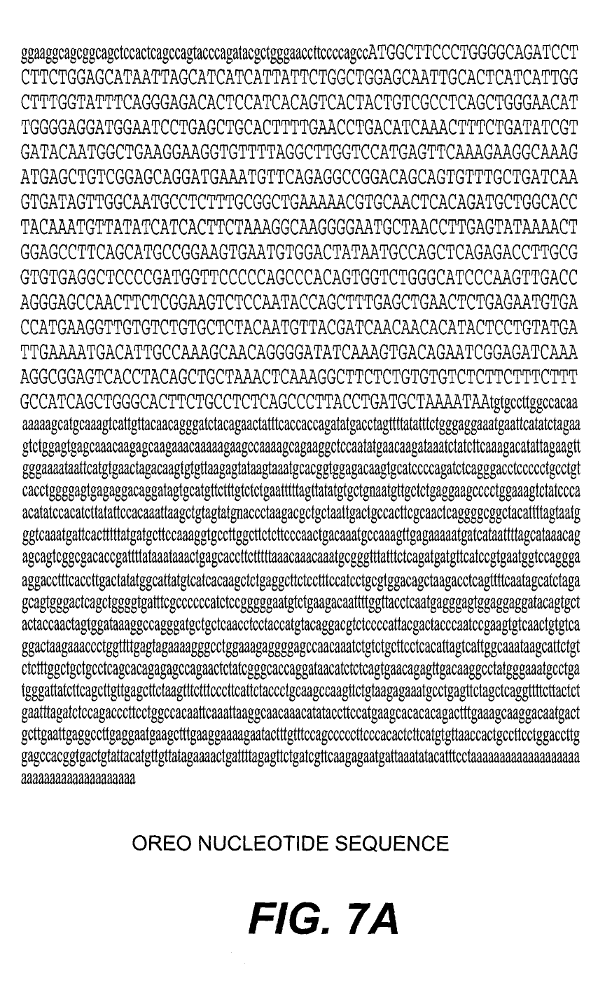

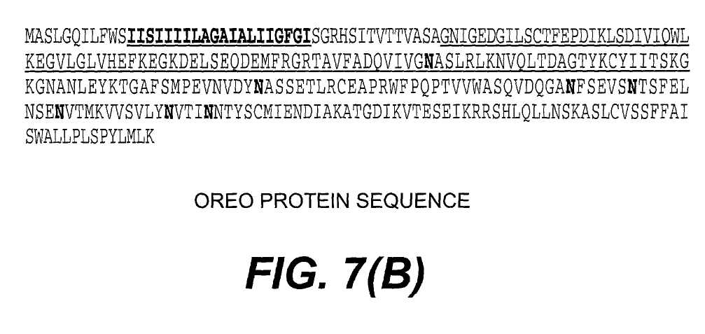

Similarly, the second gene (gene B) (“Oreo”), fragments of which gene are shown in FIGS. 4(a) and (b) and the full length in FIG. 7(a) and (b), was identified in an RDA screen where cDNA from an ovarian tumor was compared with corresponding cDNA from normal epithelial tissues (heart, liver, kidney and lung). Upon sequence analysis, it was found that this sequence matches parts of Genbank Accession # AL080312 which consists of 94644 nucleotides of a genomic sequence derived from chromosome 20, containing genetic locus 20p 11.21-11.23.

The fragment of the Oreo gene contained in FIG. 4(a) also was found to match identically an EST in the dbEST database available from the ATCC as Accession #3413715. It was found that the entire sequence is contained within this ATCC clone.

However, the fragment shown in FIG. 4(b) is not contained in this ATCC clone. As disclosed in the examples with follow, PCR amplification using primers positioned at the end of the gene fragment shown in FIG. 4(a) and the beginning of the fragment shown in FIG. 4(b), resulted in a PCR product of about 580 base points providing suggestive evidence that these sequences are contained in the same cDNA and therefore correspond to the same gene. This PCR product (which is contained in FIG. 4(e)) was also found to match identically nucleotides 72023 to 72585 of the Genbank clone having Accession # AL030812.

As described in the Examples which follow, this second gene (gene B) was found to be expressed by three out of five ovarian tumor types tested, and is also expressed by normal pancreas, kidney, ovary, prostate and testes. It is anticipated, based on these results, that this second gene will also provide a useful target for treatment and/or detection of ovarian cancers.

In particular, the use of the two novel genes of the present invention, respectively having the nucleotide sequences contained in FIG. 1(a), FIGS. 4(a) and (b), and FIG. 7(a) will be used to design novel antisense therapies for treatment of ovarian cancer, and potentially other cancers that over express either or both of these genes. Moreover, the corresponding protein shown in FIG. 7(b) will be used to produce antibodies for diagnostic and therapeutic applications.

Such therapies will involve the synthesis of oligonucleotides having sequences in the antisense orientation relative to the two novel genes identified by the present inventors. Suitable therapeutic antisense oligonucleotides will typically vary in length from two to several hundred nucleotides in length, more typically about 50-70 nucleotides in length. These antisense oligonucleotides may be administered as naked DNAs or in protected forms, e.g., encapsulated in liposomes. The use of liposomal or other protected forms may be advantageous as it may enhance in vivo stability and delivery to target sites, i.e., ovarian tumor cells.

Also, the subject novel genes may be used to design novel ribozymes that target the cleavage of the corresponding mRNAs in ovarian tumor cells. Similarly, these ribozymes may be administered in free (naked) form or by the use of delivery systems that enhance stability and/or targeting, e.g., liposomes. Ribozymal and antisense therapies used to target genes that are selectively expressed by cancer cells are well known in the art.

Also, the present invention embraces the administration of use of DNAs that hybridize to the novel gene targets identified infra, attached to therapeutic effector moieties, e.g., radiolabels, e.g., yttrium, iodine, cytotoxins, cytotoxic enzymes, in order to selectively target and kill cells that express these genes, i.e., ovarian tumor cells.

Still further, the present invention encompasses non-nucleic acid based therapies. Particularly, the invention encompasses the use of the novel cDNAs disclosed in the examples and shown in the FIGS. 1(a), 4(a) and 4(b), for expression of the corresponding antigens. It is anticipated that these antigens may be used as therapeutic or prophylactic anti-tumor vaccines. For example, a particular contemplated application of these antigens involves their administration with adjuvants that induce a cytotoxic T lymphocyte response. An especially preferred adjuvant developed by the Assignee of this application, IDEC Pharmaceuticals Corporation, is disclosed in U.S. Pat. Nos. 5,709,860, 5,695,770, and 5,585,103, the disclosures of which are incorporated by reference in their entirety. In particular, the use of this adjuvant to promote CTL responses against prostate and papillomavirus related human ovarian cancer has been suggested.

Also, administration of the subject novel antigens in combination with an adjuvant may result in a humoral immune response against such antigens, thereby delaying or preventing the development of ovarian cancer.

Essentially, these embodiments of the invention will comprise administration of one or both of the subject novel ovarian cancer antigens, ideally in combination with an adjuvant, e.g., PROVAX®, which comprises a microfluidized adjuvant containing Squalene, Tween and Pluronic, in an amount sufficient to be therapeutically or prophylactically effective. A typical dosage will range from 50 to 20,000 mg/kg body weight, have typically 100 to 5000 mg/kg body weight.

Alternatively, the subject ovarian tumor antigens may be administered with other adjuvants, e.g., ISCOMS, DETOX, SAF, Freund's adjuvant, Alum, Saponin, among others.

Yet another embodiment of the invention will comprise the preparation of monoclonal antibodies against the antigens encoded by gene A and B (contained in FIGS. 1(a), 4(a) and 4(b), and 7(a) and 7(b), respectively), the protein sequence for which gene B (Oreo) is contained in FIG. 7(b), the identification of which is identified in the Examples. Such monoclonal antibodies will be produced by conventional methods and include human monoclonal antibodies, humanized monoclonal antibodies, chimeric monoclonal antibodies, single chain antibodies, e.g., scFv's and antigen-binding antibody fragments such as Fabs, 2 Fabs, and Fab′ fragments. Methods for the preparation of monoclonal antibodies and fragments thereof, e.g., by pepsin or papain-mediated cleavage are well known in the art. In general, this will comprise immunization of an appropriate (non-homologous) host with the subject ovarian cancer antigens, isolation of immune cells therefrom, use of such immune cells to make hybridomas, and screening for monoclonal antibodies that specifically bind to either of such antigens.

These monoclonal antibodies and fragments will be useful for passive anti-tumor immunotherapy, or may be attached to therapeutic effector moieties, e.g., radiolabels, cytotoxins, therapeutic enzymes, agents that induce apoptosis, in order to provide for targeted cytotoxicity, i.e., killing of human ovarian tumor cells. Given the fact that the subject genes are apparently not significantly expressed by many normal tissues this should not result in significant adverse side effects (toxicity to non-target tissues).

In this embodiment, such antibodies or fragments will be administered in labeled or unlabeled form, alone or in combination with other therapeutics, e.g., chemotherapeutics such as progestin, EGFR, Taxol; etc. The administered composition will include a pharmaceutically acceptable carrier, and optionally adjuvants, stabilizers, etc., used in antibody compositions for therapeutic use.

Preferably, such monoclonal antibodies will bind the target antigens with high affinity, e.g., possess a binding affinity (Kd) on the order of 10⁻⁶ to 10⁻¹⁰ M.

As noted, the present invention also embraces diagnostic applications that provide for detection of the two novel genes disclosed herein. Essentially, this will comprise detecting the expression of one or both of these genes at the DNA level or at the protein level.

At the DNA level, expression of the subject genes will be detected by known DNA detection methods, e.g., Northern blot hybridization, strand displacement amplification (SDA), catalytic hybridization amplification (CHA), and other known DNA detection methods. Preferably, a cDNA library will be made from ovarian cells obtained from a subject to be tested for ovarian cancer by PCR using primers corresponding to either or both of the novel genes disclosed in this application.

The presence or absence of ovarian cancer will be determined based on whether PCR products are obtained, and the level of expression. The levels of expression of such PCR product may be quantified in order to determine the prognosis of a particular ovarian cancer patient (as the levels of expression of the PCR product likely will increase as the disease progresses.) This may provide a method of monitoring the status of an ovarian cancer patient. Of course, suitable controls will be effected.

Alternatively, the status of a subject to be tested for ovarian cancer may be evaluated by testing biological fluids, e.g., blood, urine, ovarian tissue, with an antibody or antibodies or fragment that specifically binds to the novel ovarian tumor antigens disclosed herein.

Methods for using antibodies to detect antigen expression are well known and include ELISA, competitive binding assays, etc. In general, such assays use an antibody or antibody fragment that specifically binds the target antigen directly or indirectly bound to a label that provides for detection, e.g., a radiolabel enzyme, fluorophore, etc.

Patients which test positive for the presence of the antigen on ovarian cells will be diagnosed as having or being at increased risk of developing ovarian cancer. Additionally, the levels of antigen expression may be useful in determining patient status, i.e., how far disease has advanced (stage of ovarian cancer).

As noted, the present invention provides two novel genes and corresponding antigens that correlate to human ovarian cancer. The present invention also embraces variants thereof. By “variants” is intended sequences that are at least 75% identical thereto, more preferably at least 85% identical, and most preferably at least 90% identical when these DNA sequences are aligned to the subject DNAs or a fragment thereof having a size of at least 50 nucleotides. This includes in particular allelic variants of the subject genes.

Also, the present invention provides for primer pairs that result in the amplification DNAs encoding the subject novel genes or a portion thereof in an mRNA library obtained from a desired cell source, typically human ovarian cell or tissue sample. Typically, such primers will be on the order of 12 to 50 nucleotides in length, and will be constructed such that they provide for amplification of the entire or most of the target gene.

Also, the invention embraces the antigens encoded by the subject DNAs or fragments thereof that bind to or elicit antibodies specific to the full length antigens. Typically, such fragments will be at least 10 amino acids in length, more typically at least 25 amino acids in length.

As noted, the subject genes are expressed in a majority of ovarian tumor samples tested. The invention further contemplates the identification of other cancers that express such genes and the use thereof to detect and treat such cancers. For example, the subject genes or variants thereof may be expressed on other cancers, e.g., breast, pancreas, lung or colon cancers. Essentially, the present invention embraces the detection of any cancer wherein the expression of the subject novel genes or variants thereof correlate to a cancer or an increased likelihood of cancer.

“Isolated human or non-human primate protein A or protein B protein” refers to any human or non-human primate A or B protein that is not in its normal human or primate cellular millieu. This includes by way of example compositions comprising recombinant protein A or B, pharmaceutical compositions comprising purified protein A or B, diagnostic compositions comprising purified protein A or B, and isolated protein compositions comprising protein A or B. In preferred embodiments, an isolated protein A or B will comprise a substantially pure protein, in that it is substantially free of other proteins, preferably that is at least 90% pure, that comprises the amino acid sequence contained in the figures herein or natural homologues or mutants having essentially the same sequence. A naturally occurring mutant might be found, for instance, in tumor cells expressing a gene encoding a mutated protein A or B protein sequence.

“Native human Oreo protein” refers to a protein that comprises the amino acid sequence contained FIG. 7(b).

“Native non-human Oreo protein” refers to a protein that is a non-human primate homologue of the protein having the amino acid sequence contained in FIG. 7(b). Given the phylogenetic closeness of humans to other primates, it is anticipated that human and non-human Oreo proteins will possess amino acid sequences that are highly similar, probably on the order of 95% sequence identity or higher.

“Isolated human or non-human primate Oreo or Gene A nucleic acid molecule or sequence” refers to a nucleic acid molecule that encodes human protein A or the Oreo protein which is not in its normal human cellular millieu, e.g., is not comprised in the human or non-human primate chromosomal DNA. This includes by way of example vectors that comprise a gene A or gene B nucleic acid molecule, a probe that comprises a gene A or gene B (Oreo) nucleic acid sequence directly or indirectly attached to a detectable moiety, e.g. a fluorescent or radioactive label, or a DNA fusion that comprises a nucleic acid molecule encoding gene A or gene B fused at its 5′ or 3′ end to a different DNA, e.g. a promoter or a DNA encoding a detectable marker or effector moiety. A preferred nucleic acid sequence encodes a human Oreo protein having the nucleic acid sequence in FIG. 7(a). Also included are natural homologues or mutants having substantially the same sequence. Naturally occurring homologies that are degenerate would encode the same protein as in FIG. 7(b) or that encoded by the nucleic acid sequences in 1(a), 4(a), 4(b), 4(c), 4(e) or 7(a), but would include nucleotide differences that do not change the corresponding amino acid sequence. Naturally occurring mutants might be found in tumor cells, wherein such nucleotide differences result in a mutant A or B (Oreo) protein. Naturally occurring homologues containing conservative substitutions are also encompassed.

“Variant of human or non-human primate gene A or gene B (Oreo) protein” refers to a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the corresponding native human or non-human primate gene A or gene (Oreo) protein wherein sequence identity is as defined infra. Preferably, this variant will possess at least one biological property in common with the native gene A or gene B (Oreo) human or non-human protein.

“Variant of human or non-human primate gene A or gene B (Oreo) nucleic acid molecule or sequence” refers to a nucleic acid sequence that possesses at least 90% sequence identity, more preferably at least 91%, more preferably at least 92%, even more preferably at least 93%, still more preferably at least 94%, even more preferably at least 95%, still more preferably at least 96%, even more preferably at least 97%, even more preferably at least 98% sequence identity, and most preferably at least 99% sequence identity, to the corresponding native human or non-human primate nucleic acid sequence, wherein “sequence identity” is as defined infra.

“Fragment of human or non-human primate A or B nucleic acid molecule or sequence” refers to a nucleic acid sequence corresponding to a portion of the native human gene A or gene B (Oreo) nucleic acid sequence contained in FIG. 1(a), 1(b), 4(a), 4(b), 4(c), 4(e), 7(a), or a native non-human primate gene A or gene B nucleic acid molecule, wherein said portion is at least about 50 nucleotides in length, or 100, more preferably at least 200 or 300 nucleotides in length.

“Antigenic fragments of A or B (Oreo) protein” refer to polypeptides corresponding to a fragment of protein A or protein B or a variant or homologue thereof that when used itself or attached to an immunogenic carrier that elicits antibodies that specifically bind protein A or protein (Oreo). Typically such antigenic fragments will be at least 20 amino acids in length.

Sequence identity or percent identity is intended to mean the percentage of the same residues shared between two sequences, referenced to human protein A or protein B or gene A or gene B, when the two sequences are aligned using the Clustal method [Higgins et al, Cabios 8:189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 [Dayhoff et al., in Atlas of Protein Sequence and Structure, Dayhoff, Ed., NDRF, Washington, Vol. 5, suppl. 3, p. 345, (1978)].

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human Gene A or gene B when determining percent conservation with non-human Gene A or gene B, e.g. mgene A or gene B, when determining percent conservation. Conservative amino acid changes satisfying this requirement are: R-K; E-D, Y-F, L-M; V-I, Q-H.

### Polypeptide Fragments

The invention provides polypeptide fragments of the disclosed proteins. Polypeptide fragments of the invention can comprise at least 8, more preferably at least 25, still more preferably at least 50 amino acid residues of human or non-human primate gene A or gene B, or an analogue thereof. More particularly such fragment will comprise at least 75, 100, 125, 150, 175, 200, 225, 250, 275 residues of the polypeptide encoded by gene A or gene B. Even more preferably, the protein fragment will comprise the majority of the native protein A or protein B, i.e. at least about 100 contiguous residues of protein A or protein B.

### Biologically Active Variants

The invention also encompasses biologically active mutants of protein A or protein B, which comprise an amino acid sequence that is at least 80%, more preferably 90%, still more preferably 95-99% similar to protein A or B.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant.

Human or non-human primate A and B protein variants include glycosylated forms, aggregative conjugates with other molecules, and covalent conjugates with unrelated chemical moieties. Also, A and B protein variants also include allelic variants, species variants, and muteins. Truncations or deletions of regions which do not affect the differential expression of the A and B protein gene are also variants. Covalent variants can be prepared by linking functionalities to groups which are found in the amino acid chain or at the N- or C-terminal residue, as is known in the art.

It will be recognized in the art that some amino acid sequence of the protein A and protein B proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the protein A or protein B which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255: 306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

### Fusion Proteins

Fusion proteins comprising proteins or polypeptide fragments of protein A or protein B can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of protein A or protein B or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize the amino acid sequence shown in FIG. 7(b) or encoded by the nucleotide sequences in FIG. 1(a) and (b) and 4(a) and (b), or can be prepared from biologically active variants or fragment of said protein sequence, such as those described above. The first protein segment can consist of a full-length protein A or B or a variant or fragment thereof.

As noted, these fragments may range in size from about 8 amino acids up to the full length of the protein.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence encoding an amino acid sequence contained in SEQ ID NO:7 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a sequence listing encoding protein A or B can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

Human or non-human primate protein A or protein B protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as mice, rats, guinea pigs, cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the gene A and gene B nucleic acid sequences provided herein. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence shown in the figures for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

### Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Also included are polynucleotide molecules comprising human or non-human primate gene A or gene B promoter and UTR sequences, operably linked to either protein A or protein B coding sequences or other sequences encoding a detectable or selectable marker. Such promoter and/or UTR-based constructs are useful for studying the transcriptional and translational regulation of protein A or protein B expression, and for identifying activating and/or inhibitory regulatory proteins.

### Host Cells

An expression construct can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad Sci. USA 80: 21-25 (1983); and Siebenlist et al., Cell 20: 269 (1980).

Expression systems in yeast include those described in Hinnnen et al., Proc. Natl. Acad. Sci. USA 75: 1929 (1978); Ito et al., J Bacteriol 153: 163 (1983); Kurtz et al., Mol. Cell. Biol. 6: 142 (1986); Kunze et al., J Basic Microbiol. 25: 141 (1985); Gleeson et al, J. Gen. Microbiol. 132: 3459 (1986), Roggenkamp et al., Mol. Gen. Genet. 202: 302 (1986)); Das et al., J Bacteriol. 158: 1165 (1984); De Louvencourt et al., J Bacteriol. 154:737 (1983), Van den Berg et al., Bio/Technology 8: 135 (1990); Kunze et al., J. Basic Microbiol. 25: 141 (1985); Cregg et al., Mol. Cell. Biol. 5: 3376 (1985); U.S. Pat. No. 4,837,148; U.S. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al, Ann. Rev. Microbiol. 42: 177 (1988); Carbonell et al., Gene 73: 409 (1988); Maeda et al., Nature 315: 592-594 (1985); Lebacq-Verheyden et al., Mol. Cell Biol. 8: 3129 (1988); Smith et al, Proc. Natl. Acad. Sci. USA 82: 8404 (1985); Miyajima et al, Gene 58: 273 (1987); and Martin et al., DNA 7:99 (1988). Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761(1985); Gorman et al., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979); Barnes and Sato, Anal. Biochem. 102: 255 (1980); U.S. Pat. No. 4,767,704; U.S. Pat. No. 4,657,866; U.S. Pat. NO. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides of the nucleotide sequence shown in the figures herein. The transcription unit is located upstream to a coding sequence of the endogenous gene. The exogenous regulatory sequence directs transcription of the coding sequence of the endogenous gene.

Human or non-human primate protein A or protein B can also include hybrid and modified forms thereof including fusion proteins, fragments and hybrid and modified forms in which certain amino acids have been deleted or replaced, modifications such as where one or more amino acids have been changed to a modified amino acid or unusual amino acid.

Also included within the meaning of substantially homologous is any human or non-human primate protein A or B which may be isolated by virtue of cross-reactivity with antibodies to the gene A or gene B described herein or whose encoding nucleotide sequences including genomic DNA, mRNA or cDNA may be isolated through hybridization with the complementary sequence of genomic or subgenomic nucleotide sequences or cDNA of the gene A or gene B herein or fragments thereof. It will also be appreciated by one skilled in the art that degenerate DNA sequences can encode human or non-human primate protein A or protein B and these are also intended to be included within the present invention as are allelic variants of gene A or gene B or non-human primate gene A or gene B.

Preferred is protein A or protein B prepared by recombinant DNA technology. By “pure form” or “purified form” or “substantially purified form” it is meant that a protein A or protein B composition is substantially free of other proteins which are not protein A or protein B.

The present invention also includes therapeutic or pharmaceutical compositions comprising protein A or protein B or non-human primate protein A or protein B in an effective amount for treating patients with disease, and a method comprising administering a therapeutically effective amount of protein A or protein B. These compositions and methods are useful for treating cancers associated with protein A or protein B, e.g. ovarian cancer. One skilled in the art can readily use a variety of assays known in the art to determine whether protein A or protein B would be useful in promoting survival or functioning in a particular cell type.

In certain circumstances, it may be desirable to modulate or decrease the amount of protein A or protein B expressed. Thus, in another aspect of the present invention, gene A or gene B anti-sense oligonucleotides can be made and a method utilized for diminishing the level of expression of protein A or protein B by a cell comprising administering one or more gene A or gene B anti-sense oligonucleotides. By gene A or gene B anti-sense oligonucleotides reference is made to oligonucleotides that have a nucleotide sequence that interacts through base pairing with a specific complementary nucleic acid sequence involved in the expression of gene A or gene B such that the expression of gene A or gene B is reduced. Preferably, the specific nucleic acid sequence involved in the expression of gene A or gene B is a genomic DNA molecule or mRNA molecule that encodes gene A or gene B. This genomic DNA molecule can comprise regulatory regions of the gene A or gene B, or the coding sequence for mature gene A or gene B protein.

The term complementary to a nucleotide sequence in the context of gene A or gene B antisense oligonucleotides and methods therefor means sufficiently complementary to such a sequence as to allow hybridization to that sequence in a cell, i.e., under physiological conditions. The gene A or gene B antisense oligonucleotides preferably comprise a sequence containing from about 8 to about 100 nucleotides and more preferably the antisense oligonucleotides comprise from about 15 to about 30 nucleotides. The gene A or gene B antisense oligonucleotides can also contain a variety of modifications that confer resistance to nucleolytic degradation such as, for example, modified internucleoside linages [Uhlmann and Peyman, Chemical Reviews 90:543-548 (1990); Schneider and Banner, Tetrahedron Lett. 31:335, (1990) which are incorporated by reference], modified nucleic acid bases as disclosed in 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T.I.B.S. 23:45-50 (1998)].

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

Additionally, protein A or protein B or non-human primate protein A or protein B can also be linked or conjugated with agents that provide desirable pharmaceutical or pharmacodynamic properties. For example, protein A or protein B can be coupled to any substance known in the art to promote penetration or transport across the blood-brain barrier such as an antibody to the transferrin receptor, and administered by intravenous injection (see, for example, Friden et al., Science 259:373-377 (1993) which is incorporated by reference). Furthermore, the subject protein A or protein B can be stably linked to a polymer such as polyethylene glycol to obtain desirable properties of solubility, stability, half-life and other pharmaceutically advantageous properties. [See, for example, Davis et al., Enzyme Eng. 4:169-73 (1978); Buruham, Am. J. Hosp. Pharm. 51:210-218 (1994) which are incorporated by reference].

The compositions are usually employed in the form of pharmaceutical preparations. Such preparations are made in a manner well known in the pharmaceutical art. See, e.g. Remington Pharmaceutical Science, 18th Ed., Merck Publishing Co. Eastern Pa., (1990). One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The subject human or primate gene A or gene B protein, fragment or variant thereof can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing the subject protein A or protein B or variant or fragment thereof are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, protein A or protein B may be therapeutically administered by implanting into patients vectors or cells capable of producing a biologically-active form of protein A or protein B or a precursor of protein A or protein B, i.e., a molecule that can be readily converted to a biological-active form of protein A or protein B by the body. In one approach, cells that secrete protein A or protein B may be encapsulated into semipermeable membranes for implantation into a patient. The cells can be cells that normally express protein A or protein B or a precursor thereof or the cells can be transformed to express protein A or protein B or a precursor thereof. It is preferred that the cell be of human origin and that the protein A or protein B be human protein A or protein B when the patient is human. However, it is anticipated that non-human primate protein A or protein B may be effective.

In a number of circumstances it would be desirable to determine the levels of protein A or protein B or corresponding mRNA in a patient. The identification of human protein A or protein B herein along with the previous evidence expression that suggests that protein A or protein B may be expressed at different levels during some diseases, e.g., cancers, provides the basis for the conclusion that the presence of gene A or gene B serves a normal physiological function related to cell growth and survival. Endogenously produced human protein A or protein B may also play a role in certain disease conditions.

The term “detection” as used herein in the context of detecting the presence of gene A or gene B in a patient is intended to include the determining of the amount of protein A or protein B or the ability to express an amount of protein A or protein B in a patient, the estimation of prognosis in terms of probable outcome of a disease and prospect for recovery, the monitoring of the protein A or protein B levels over a period of time as a measure of status of the condition, and the monitoring of protein A or protein B levels for determining a preferred therapeutic regimen for the patient, e.g. one with ovarian cancer.

To detect the presence of gene A or gene B in a patient, a sample is obtained from the patient. The sample can be a tissue biopsy sample or a sample of blood, plasma, serum, CSF or the like. It has been found that gene A or gene B is expressed at high levels in some cancers, e.g., colon, breast and lung cancer. Samples for detecting protein A or protein B can be taken from these tissue. When assessing peripheral levels of protein A or protein B, it is preferred that the sample be a sample of blood, plasma or serum. When assessing the levels of protein A or protein B in the central nervous system a preferred sample is a sample obtained from cerebrospinal fluid or neural tissue.

In some instances, it is desirable to determine whether the gene A or gene B is intact in the patient or in a tissue or cell line within the patient. By an intact gene A or gene B, it is meant that there are no alterations in the gene such as point mutations, deletions, insertions, chromosomal breakage, chromosomal rearrangements and the like wherein such alteration might alter production of gene A or gene B or alter its biological activity, stability or the like to lead to disease processes. Thus, in one embodiment of the present invention a method is provided for detecting and characterizing any alterations in the gene A or gene B. The method comprises providing an oligonucleotide that contains the gene A or gene B cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize specifically to the A or B gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact A or B gene or an A or B gene abnormality.

Hybridization to an A or B gene would involve denaturing the chromosomal DNA to obtain a single-stranded DNA; contacting the single-stranded DNA with a gene probe associated with the gene A or gene B gene sequence; and identifying the hybridized DNA-probe to detect chromosomal DNA containing at least a portion of a human gene A or gene B.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The gene A or gene B probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 250-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

Gene A or gene B abnormalities can also be detected by using the PCR method and primers that flank or lie within the gene A or gene B gene. The PCR method is well known in the art. Briefly, this method is performed using two oligonucleotide primers which are capable of hybridizing to the nucleic acid sequences flanking a target sequence that lies within an A or B gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising gene A or gene B or a fragment thereof is then directly sequenced and analyzed by comparison of the sequence with the sequences disclosed herein to identify alterations which might change activity or expression levels or the like.

In another embodiment, a method for detecting protein A or protein B is provided based upon an analysis of tissue expressing the gene A or gene B. Certain tissues such as breast, lung, colon and others have been found to express the gene A or gene B. The method comprises hybridizing a polynucleotide to mRNA from a sample of tissue that normally expresses the gene A or gene B. The sample is obtained from a patient suspected of having an abnormality in the gene A or gene B.

To detect the presence of mRNA encoding protein A or protein B, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding protein A or protein B or a derivative of the cDNA as a probe, high stringency conditions can be used in order to prevent false positives, that is the hybridization and apparent detection of gene A or gene B nucleotide sequences when in fact an intact and functioning gene A or gene B gene is not present. When using sequences derived from the gene A or gene B cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. [Sambrook et al. (1989), supra].

In order to increase the sensitivity of the detection in a sample of mRNA encoding the protein A or protein B, the technique of reverse transcription/polymerization chain reaction (RT/PCR) can be used to amplify cDNA transcribed from mRNA encoding the protein A or protein B. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and gene A or gene B specific primers. [Belyavsky et al., Nucl. Acid Res. 17:2919-2932 (1989); Krug and Berger, Methods in Enzymology, 152:316-325, Academic Press, NY (1987) which are incorporated by reference].

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the protein A or protein B in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. [Basic and Clinical Immunology, 217-262, Sites and Terr, eds., Appleton & Lange, Norwalk, Conn., (1991), which is incorporated by reference]. Preferred are binder-ligand immunoassay methods including reacting antibodies with an epitope or epitopes of the gene A or gene B protein and competitively displacing a labeled gene A or gene B protein or derivative thereof.

As used herein, a derivative of the protein A or protein B is intended to include a polypeptide in which certain amino acids have been deleted or replaced or changed to modified or unusual amino acids wherein the derivative is biologically equivalent to gene A or gene B and wherein the polypeptide derivative cross-reacts with antibodies raised against the protein A or protein B. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioinununoassay (RIA), enzyme immunoassays, e.g., enzyme-linked immunosorbent assay (ELISA), fluorescent immunoassays and the like.

Polyclonal or monoclonal antibodies to the subject non-human primate or human gene A or gene B protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, typically a rabbit, hamster or a mouse.

Oligopeptides can be selected as candidates for the production of an antibody to the gene A or gene B protein based upon the oligopeptides lying in hydrophilic regions, which are thus likely to be exposed in the mature protein. Peptide sequence used to generate antibodies against gene A or gene B include:

Additional oligopeptides can be determined using, for example, the Antigenicity Index, Welling, G. W. et al., FEBS Lett. 188:215-218 (1985), incorporated herein by reference.

In other embodiments of the present invention, humanized monoclonal antibodies are provided, wherein the antibodies are specific for protein A or protein B. The phrase “humanized antibody” refers to an antibody derived from a non-human antibody, typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3): 169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which patents are incorporated herein by reference.

Humanized antibodies to protein A or protein B can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNF, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, protein A or protein B and variants thereof are used to immunize a transgenic animal as described above. Monoclonal antibodies are made using methods known in the art, and the specificity of the antibodies is tested using isolated protein A or protein B.

Methods for preparation of the human or primate gene A or gene B protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system [E.I. du Pont de Nemours Company, Wilmington, Del.) (Caprino and Han, J. Org. Chem. 37:3404 (1972) which is incorporated by reference].

Polyclonal antibodies can be prepared by immunizing rabbits or other animals by injecting antigen followed by subsequent boosts at appropriate intervals. The animals are bled and sera assayed against purified protein A or protein B usually by ELISA or by bioassay based upon the ability to block the action of gene A or gene B or primate gene A or gene B. In a non-limiting example, an antibody to gene A or gene B can block the binding of gene A or gene B to Dishevelled protein. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. [Milstein and Kohler, Nature 256:495-497 (1975); Gulfre and Milstein, Methods in Enzymology: Immunochemical Techniques 73:1-46, Langone and Banatis eds., Academic Press, (1981) which are incorporated by reference]. The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the A or B protein by treatment of a patient with specific antibodies to the A or B protein.

Specific antibodies, either polyclonal or monoclonal, to the A or B protein can be produced by any suitable method known in the art as discussed above. For example, murine or human monoclonal antibodies can be produced by hybridoma technology or, alternatively, the A or B protein, or an immunologically active fragment thereof, or an anti-idiotypic antibody, or fragment thereof can be administered to an animal to elicit the production of antibodies capable of recognizing and binding to the A or B protein. Such antibodies can be from any class of antibodies including, but not limited to IgG, IgA, IgM, IgD, and IgE or in the case of avian species, IgY and from any subclass of antibodies.

The availability of isolated human or primate protein A or protein B allows for the identification of small molecules and low molecular weight compounds that inhibit the binding of protein A or protein B to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbence, fluorescence or luminescence as read-outs. [Gonzalez, J. E. et al., Curr. Opin. Biotech. 9:624-63 1(1998)].

Model systems are available that can be adapted for use in high throughput screening for compounds that inhibit the interaction of protein A or protein B with its ligand, for example by competing with protein A or protein B for ligand binding. Sarubbi et al., Anal. Biochem. 237:70-75 (1996) describe cell-free, non-isotopic assays for discovering molecules that compete with natural ligands for binding to the active site of IL-1 receptor. Martens, C. et al., Anal. Biochem. 273:20-31 (1999) describe a generic particle-based nonradioactive method in which a labeled ligand binds to its receptor immobilized on a particle; label on the particle decreases in the presence of a molecule that competes with the labeled ligand for receptor binding.

The therapeutic gene A or gene B polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic according to the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/3 0763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63: 3822-3828 (1989); Mendelson et al., Virol. 166: 154-165 (1988); and Flotte et al., P.N.A.S. 90: 10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisler et al., P.N.A.S. 90: 11498-11502 (1993); Guzman et al., Circulation 88: 2838-2848 (1993); Guzman et al., Cir. Res. 73: 1202-1207 (1993); Zabner et al., Cell 75: 207-216 (1993); Li et al., Hum. Gene Ther. 4: 403-409 (1993); Cailaud et al., Eur. J. Neurosci. 5: 1287-1291 (1993); Vincent et al., Nat. Genet. 5: 130-134 (1993); Jaffe et al., Nat. Genet. 1: 372-378 (1992); and Levrero et al., Gene 101: 195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3: 147-154 (1992) may be employed.

Other gene delivery vehicles and methods may be employed, including polycationic condensed DNA linked or unlinked to killed adenovirus alone, for example Curiel, Hum. Gene Ther. 3: 147-154 (1992); ligand-linked DNA, for example see Wu, J. Biol. Chem. 264: 16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Pat. Publication Nos. WO 95/13 796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

While the invention has been described supra, including preferred embodiments, the following examples are provided to further illustrate the invention.

### EXAMPLE 1

### Identification of Gene A

This gene was identified in an RDA screen where cDNA derived from an ovarian tumor was compared with corresponding material derived from the matched normal ovary. FIG. 1 (a) depicts the sequence identified by the RDA screen as being overexpressed in the ovarian tumor. This sequence was searched against Genbank databases and was found to match an EST in the dbEST database. This database is public domain, accessible through the NIH. The accession number for this EST is AA133536. This EST is available to the public through the ATCC and is listed in the ATCC Catalog as clone # 947812. This clone was ordered from ATCC and sequenced completely. The sequence of the clone is shown in 1(b). Translation of this sequence yields a 786 nucleotide continuous open reading frame, followed by 454 nucleotides of 3′ UTR ending in a poly A tail. This indicates that the complete 3′ end of gene A is contained within this ATCC clone. This sequence information will now be used to design gene specific PCR primers in order to PCR the full length sequence of Gene A from a cDNA population. This cDNA is commercially available from Clontech and is sold under the name ‘Marathon-Ready cDNA’. cDNA from human placenta, Clontech catalog # 7411-1 will be used as the starting material to clone gene A.

### EXAMPLE 2

### Expression of Gene A in Normal Tissues

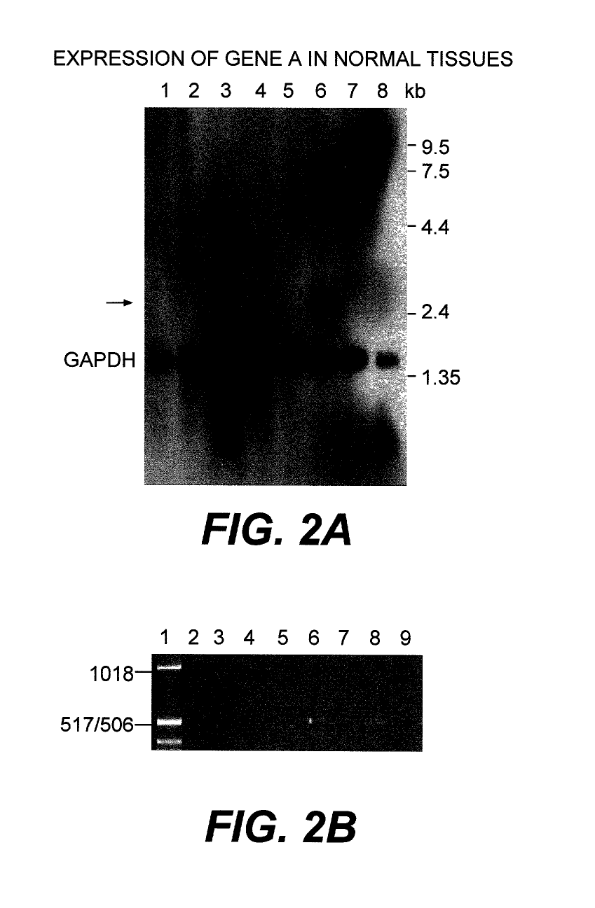

As shown in FIG. 2(a), the expression of gene A was measured by multiple tissue northern blot. A poly A+ blot (Clontech MTN blot, catalog #7760-1) was probed with the gene A sequence depicted in 1(a). A faint band approximately 2.5 kb in size can be seen in lane 6 which contains placenta mRNA. This data indicates that a single 2.5 kb transcript of this gene is expressed in placenta, but not in any of the other tissues on this blot. As a control, the blot was also probed for Glyceraldehyde 3-phosphate dehydrogenase (GAPDH), a housekeeping gene expressed highly in all tissues. The location of the GAPDH message is indicated on the figure. RNA samples in the lanes are as follows: 1, pancreas; 2, kidney; 3, skeletal muscle; 4, liver; 5, lung; 6, placenta; 7, brain; 8, heart.

FIG. 2(b) shows the expression of gene A measured by PCR. In this experiment, primers contained within the sequence depicted in 1(b) were used to detect gene A expression in a panel of cDNAs prepared from additional normal tissues (Clontech MTC panel, catalog # K1421-1.) The anticipated 502 bp PCR product was found only in lane 8, which contained testis cDNA. Lanes on the gel are as follows: 1, DNA size markers; 2, colon; 3, ovary; 4, peripheral blood leukocytes; 5, prostate; 6, small intestine; 7, spleen; 8, testis; 9, thymus.

Primer sequences for MTN blot:

5′-ACGTGCGCGCCCACGGAGGCC-3′

5′-AGAGGCAGGCGGGGGCAAAGC-3′

Primer sequences for PCR:

5′-CCTGGACAACAAGCAGTATCGCC-3′

5′-GACGGGGCAGCCCTTGAGGG-3′

Taken together, the data presented in FIG. 2(a) and 2(b) indicate that gene A is expressed only in normal placenta and testis. When an MTC panel corresponding to the tissues in the MTN blot in panel A was examined, faint PCR products were observed in liver and kidney in addition to the placenta (data not shown.) This may indicate low level expression of the gene in these organs. Further experiments using the full length sequence will determine whether such tissues express this gene.

### EXAMPLE 3

### Expression of Gene A in Tumor Tissues

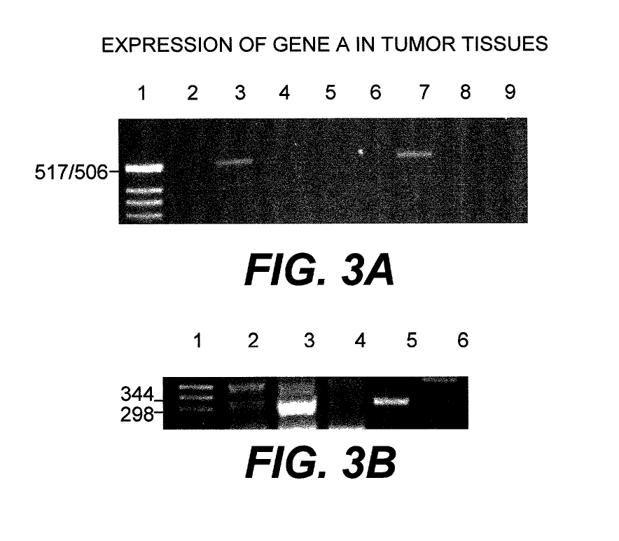

FIG. 3(a) shows the results of an experiment that measured expression in a panel of human tumors. The primer pair used for the PCR experiment in FIG. 2(a) and (b) was used to examine gene A expression in a panel of cDNAs prepared from 8 different human tumors (Clontech MTC panel, catalog # K1422-1.) The anticipated 502 bp product was observed in lanes 3 and 7, corresponding to lung and colon carcinomas. Gel lanes are as follows: 1, DNA size markers; 2, breast carcinoma; 3, lung carcinoma (a); 4, colon adenocarcinoma (a); 5, lung carcinoma (b); 6, prostate adenocarcinoma; 7, colon adenocarcinoma (b); 8, ovary carcinoma; 9, pancreatic adenocarcinoma.

FIG. 3(b) contains the results of a PCR experiment that measured gene A expression in a panel of ovarian tumors. The primer pair used for the MTN blot in FIG. 2(a) and 2(b) was used to examine expression of gene A in cDNA samples prepared from 4 different ovarian tumors (lanes 2-4, Biochain multi-sample cDNA panel, catalog # 0546161; lanes 5-6 Clontech ovary matched pair, catalog # HP101O.) The anticipated 313 bp product was detected in 3 of 4 ovarian samples on the gel. The upper band seen in some of the gel lanes is a nonspecific PCR artifact.

Gel lanes are as follows: 1, DNA size markers; 2, poorly differentiated adenocarcinoma; 3, cystadenocarcinoma; 4, cystadenoma; 5, serous cystadenocarcinoma; 6, normal ovary.

Thus, the results in Examples 1 through 3 and the Figures disclosed therein indicate that:

(i) gene A is expressed highly in normal placenta, but is absent or barely detectable in all other normal tissues examined.

(ii) gene B is expressed in 3 out of 5 ovarian tumor types examined. This suggests that this gene may provide a suitable tumor marker or target for ovarian cancer therapy.

### EXAMPLE 4

### Identification of A Second Ovarian Cancer Gene (Gene B)(Oreo)

A second ovarian cancer gene, gene B (Oreo gene), was also identified in an RDA screen wherein cDNA derived from an ovarian tumor was compared with corresponding cDNA derived from an equal mixture of normal epithelial tissues (heart, liver, kidney and lung.) FIGS. 4(a) and (b) contain the sequence fragments of gene B identified in the RDA screen. When these sequences were compared against Genbank databases, they were found to match part of Genbank accession # AL080312. This entry consists of 94644 nucleotides of genomic sequence from chromosome 20, comprising the genetic locus 20p11.21-11.23. The sequence in (a) matches nucleotides 71731-72066 in the Genbank sequence, while (b) matches nucleotides 72564-72876. The recently updated Genbank database now assigns the Oreo gene to chromosome 1, and the original assignment on chromosome 20 was incorrect.

The sequence fragment in (a) was also found to match an EST in the dbEST database. The EST is designated AI799522 in Genbank, and was obtained from the ATCC, where it is listed under catalog # 3413715. Complete sequencing of the ATCC clone provided the information shown in FIG. 4 (c). The fragment originally identified in (a) was contained completely within the sequence of this ATCC clone and is shown in bold text in panel (c).

Recently, there have been three other Genbank entries corresponding to the “Oreo” sequence. The most recent is XM_{—}018334, a direct sequence submission from the NCBT on Apr. 16, 2001. The entry lists the gene as “Homo sapiens hypothetical protein FLJ22418.” The other submissions are AK026071, submitted Sep. 29, 2000, and NM_{—}024626 submitted on Mar. 18, 2001. These submissions are both from the Human Genome Center, Institute of Medical Science, University of Tokyo.

Translation of the sequence in (c) yields a 924 nucleotide continuous open reading frame, followed by 102 nucleotides of 3′ UTR.

The fragment originally identified in (b) is not part of the EST clone. It is believed that the ATCC clone does not contain the full 3′ UTR, and the sequence in (b) is 3′ UTR located further downstream of the end of this clone. Experimental evidence in support of this hypothesis is shown in the FIG. 4(d). In this experiment, ovarian cDNA used in the initial RDA screen was used as template in a PCR reaction containing primers positioned at the end of the gene fragment shown in 4(a) and the beginning of the fragment shown in 4(b). Primer sequences are underlined in the figure. A PCR product approximately 580 bp in size was obtained in the reaction, indicating that these 2 DNA sequences are part of the same cDNA. This PCR product was cloned and sequenced, the results of which are presented in FIG. 4(e). The cloned product matches nucleotides 72023-72585 in Genbank entry AL030812. The primers used to PCR this product are underlined in panels (a), (b) and (e). This result indicates that the DNA fragment presented in (b) is in fact part of the same gene.

### EXAMPLE 5

### Expression of Gene B in Normal Tissues

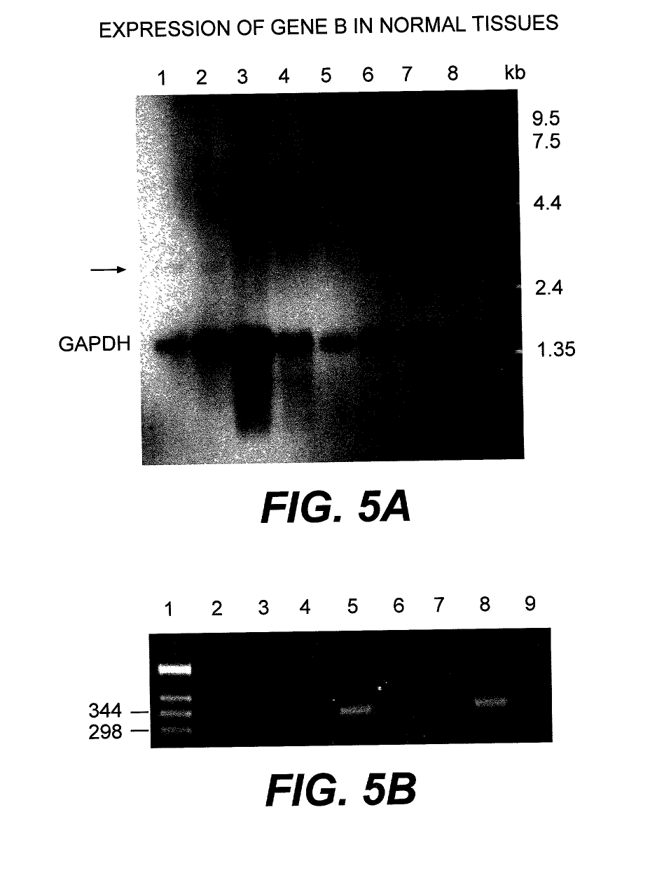

FIG. 5(a), contains an experiment wherein expression of gene B was measured by multiple tissue northern blot. A poly A+ blot (Clontech MTN blot, catalog # 7760-1) was probed with the Gene B sequence depicted in FIG. 4(b). A faint band approximately 2.5 kb in size can be seen in lanes 1 & 2, as indicated by the arrow. The blot was also probed with a GAPDH probe as a control. The location of the GAPDH message is indicated in the figure. RNA samples in the lanes are as follows: 1, pancreas; 2, kidney; 3, skeletal muscle; 4, liver; 5, lung; 6, placenta; 7, brain; 8, heart.

FIG. 5(b) contains the results of an experiment wherein gene B expression was measured by PCR. Primers contained within the sequence depicted in FIG. 4(b) were used to detect gene B expression in a panel of cDNAs prepared from additional normal tissues (Clontech MTC panel, catalog # K1421-1.) The expected 307bp product was found only in lanes 3, 5 and 8, which correspond to ovary, prostate and testis cDNA, indicating the gene is expressed only in these organs. Samples are as follows: 1, DNA size markers; 2, colon; 3, ovary; 4, peripheral blood leukocytes; 5, prostate; 6, small intestine; 7, spleen; 8, testis; 9, thymus. The primers used are set forth below.

PCR primers:

5′-TCCCCTGCCTGTCACCTGGGG-3′

5′-TTTTTCTCAACTTTGGCATTTG-3′

The data presented in this figure indicates that gene B is expressed in pancreas, kidney, ovary, prostate and testis. Expression in the latter 3 organs does not present a problem in terms of developing a therapeutic to target this gene. The extent of gene expression in kidney and pancreas however will need to be reevaluated once the full length gene is cloned.

### EXAMPLE 6

### Expression of Gene B in Tumor Tissues

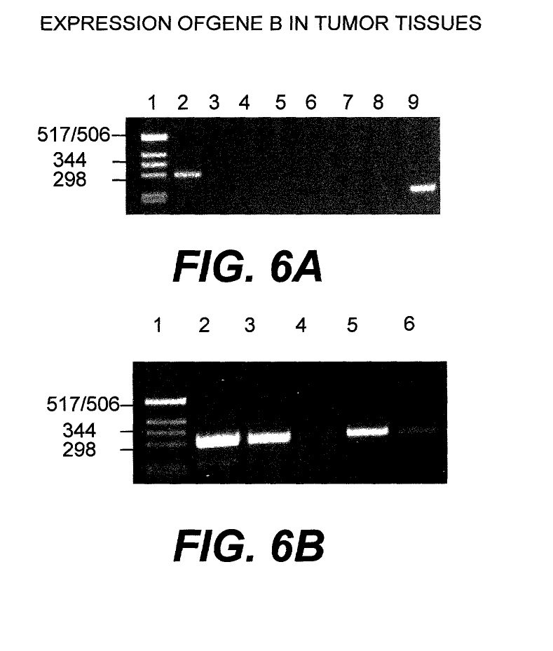

FIG. 6(a), contains the results of an experiment wherein the expression of gene B was evaluated in a panel of human tumors. Specifically, the primer pair described in FIG. 5 was used to examine gene B expression in a panel of cDNAs prepared from 8 different human tumors (Clontech MTC panel, catalog # K1422-1). A 307 bp PCR product was observed in lanes 2 and 9, corresponding to breast and pancreas carcinomas. Gel lanes are as follows: 1, DNA size markers; 2, breast carcinoma; 3, lung carcinoma; 4, colon adenocarcinoma; 5, lung carcinoma; 6, prostate adenocarcinoma; 7, colon adenocarcinoma; 8, ovary carcinoma; 9, pancreatic adenocarcinoma.

FIG. 6(b) Gene expression in a panel of ovarian tumors. The same primer pairs were used to examine gene B expression in cDNA samples prepared from 4 different ovarian tumors (lanes 2-4, Biochain multi-sample cDNA panel, catalog # 0546161; lane 5-6 Clontech ovary matched pair, catalog # HP101O. ) The anticipated 307 bp product was detected in 3 of 4 ovarian tumor samples on the gel.

Gel lanes are as follows: 1, DNA size markers; 2, poorly differentiated adenocarcinoma; 3, cystadenocarcinoma; 4, cystadenoma; 5, serous cystadenocarcinoma; 6, normal ovary.

Taken together, the data presented in FIG. 6 indicate that gene B is expressed in three out of five different ovarian tumor samples analyzed. This suggests that this gene will provide a suitable tumor marker or target for detection or treatment of ovarian cancer, and potentially other cancers.

### EXAMPLE 7

### Characterization of Structure of Oreo Gene and Protein

FIG. 7(a) shows the complete nucleotide sequence of Oreo. The Oreo gene comprises a 2.62 kb cDNA, containing a 60 bp 5′ untranslated sequence, 846 bp open reading frame and 1.7 kb 3′ untranslated region. The open reading frame is shown in bold and upper case in the figure. The complete ORF, 5′ untranslated sequence and the first 183 nucleotides of the 3′ untranslated sequence were obtained by sequencing the EST designated as Genbank accession # AI799522, ATCC catalog # 3413715. The remainder of the 3′ untranslated sequence was obtained by Rapid Amplification of cDNA Ends (RACE), using the 3′ RACE System from Life Technologies (catalog # 18373-019).

### EXAMPLE 8

### Characterization of Structure of Oreo Protein

FIG. 7(b) shows the predicted amino acid sequence of the open reading frame indicated in 7(a). This ORF codes for a 282 amino acid protein with a predicted molecular weight of 30.9 kD. The sequence contains a predicted transmembrane domain shown in bold and underlined, a predicted Ig domain which is underlined, and 7 potential N-linked glycosylation sites shown in bold. Based on these motifs, Oreo is predicted to be a type 2 transmembrane glycoprotein, oriented with an extracellular C-terminal portion and intracellular N-terminal portion. The presence of an Ig domain suggests that Oreo may be a cell surface receptor.

### EXAMPLE 9

### Genomic Arrangement of Oreo Gene and Protein Domains

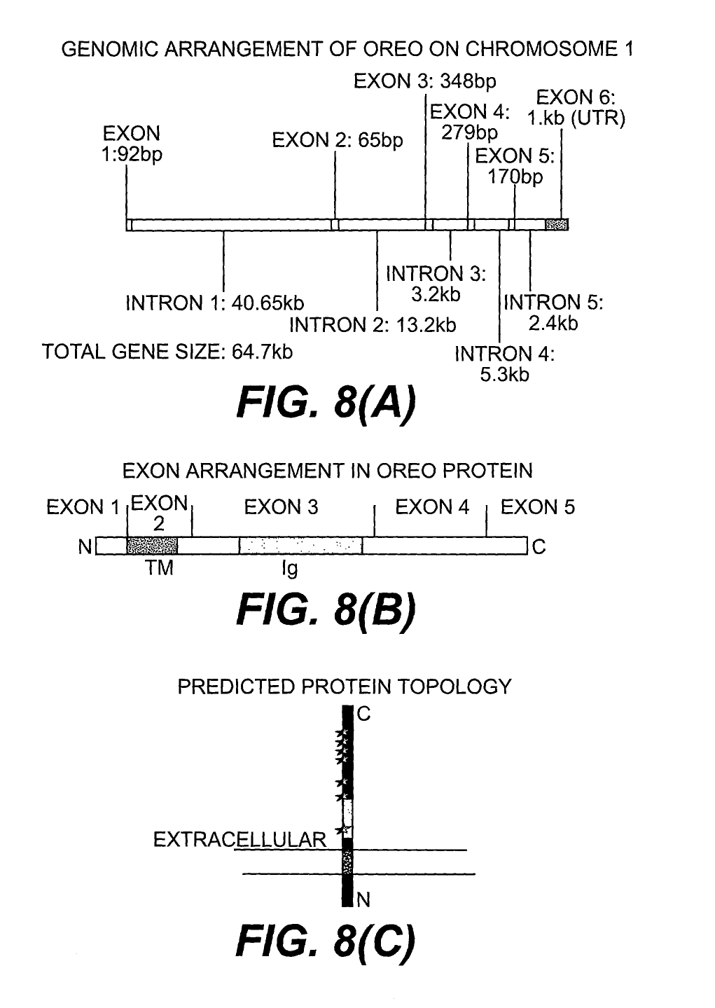

FIG. 8 (a) depicts the genomic arrangement of the Oreo gene. This arrangement was determined by analysis of a single contig of chromosome 1, clone RP11-229Al9. This clone has been assigned Genbank # AL391476. The Oreo gene comprises 6 exons spanning a region of 64.7 kb on the chromosome. The exons (coding regions) are depicted in red in the figure, introns are in blue.

FIG. 8(b) depicts Oreo exon arrangement as it relates to predicted protein domains. The transmembrane domain is shown in pink, the Ig domain in green.

### EXAMPLE 10

### Expression of Oreo on Plasma Membrane

FIG. 8(c) is a schematic showing the predicted topology of Oreo in the plasma membrane. The transmembrane domain is shown in pink, the Ig domain in green, and the potential glycosylation sites are depicted by red stars.

The data in Table 1 below provides information on the expression of Oreo in various tissues as determined using the Gene Logic GeneExpress™ Oncology Datasuite. The first column lists the percentage of each tissue type expressing Oreo. The median expression level measured for each tissue type is reported in the next column, while the last column lists the range of expression. The total number of samples for each tissue type is as follows: ovary tumor, 13; normal ovary, 20; normal kidney, 19; normal liver, 20; normal lung, 21; normal colon, 25; normal pancreas, 10; normal breast, 19. The expression values were obtained directly from the Oncology DataSuite and were determined using Gene Logic's proprietary normalization algorithm. A median entry with no range indicates expression was detectable in a single sample of the corresponding tissue type.

TABLE 1
Oreo Expression Levels In Ovarian Tumors and Normal Tissues As
Determined Using The Gene Logic GeneExpress ™ Oncology Datasuite

| | Expression Data | | |
|---|---|---|---|
| | | Median | Range of |
| | Expression, | Expression | Expression |
| | % if tissue | (Gene Logic | (Gene Logic |
| Tissue | samples | units) | units) |
| Ovary Tumor | 69 | 265 | 25-2214 |
| Normal Ovary | 10 | 44 | 43-44 |
| Normal Breast | 79 | 290 | 25-1238 |
| Normal Kidney | 58 | 124 | 44-391 |
| Normal Liver | 15 | 92 | 21-112 |
| Normal Lung | 14 | 95 | 61-134 |
| Normal Colon | 4 | 77 | — |
| Normal Pancreas | 10 | 51 | 17-84 |

### EXAMPLE 11

### Expression of Oreo by Ovarian Tumor and Normal Cell Lines Determined by Northern Analyses

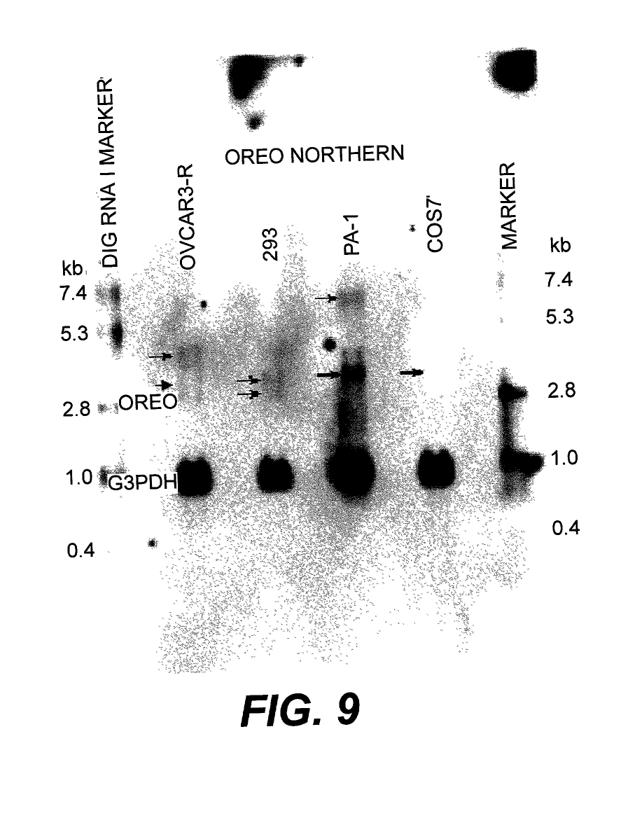

FIG. 9: Oreo expression in ovarian tumor and normal cell lines as measured by Northern blot analysis.

Ten μg of total RNA from HEK293 (human embryonic kidney cell line), Cos-7 (African green monkey kidney cell line) and ovarian tumor lines Ovcar-3 and PA-1 were probed with a digoxygenin labeled probe corresponding to nucleotides 469-769 of the Oreo gene. As a control, the blot was also probed for Glyceraldehyde 3 phosphate dehydrogenase (GAPDH), a housekeeping gene expressed at high levels in all cell lines and tissues. The location of both the GAPDH and Oreo messages are indicated on the figure. Arrowheads in the figure indicate the presence of multiple Oreo transcripts in the cell lines.

### EXAMPLE 12

### Indirect immunofluorescence Localization of Oreo

The cellular localization of Oreo was determined in the following experiment. Two Oreo constructs were prepared in the mammalian expression vector pcDNA3.1 (Invitrogen, catalog #V810-20 ). One construct comprised the full coding sequence of Oreo, hereafter referred to as ‘intact Oreo’; the second comprised only the predicted extracellular domain, referred to as ‘Oreo-exo’. Both constructs were created in frame with the C-terminal V5 epitope contained in the vector. A Kozak sequence with initiator methionine was added at the start of OREO exo. The DNA elements comprising the pcDNA3.1 vector are detailed in the Invitrogen catalog.

Both Oreo constructs were transfected into COS-7 cells grown on coverslips in 6 well tissue culture dishes. Transfections were carried out using Lipofectamine Reagent (Life Technologies, catalog # 10964-013) and 1.5 μg plasmid DNA per well. Transfection medium consisted of 6 μL Lipofectamine mixed with 2 mL Opti-MEM medium(Life Technologies, catalog # 31985-070) and plasmid DNA. Cells were incubated in the mixture at 37° C. for 5 hours, after which time an equal volume of medium containing 20% FBS (HyClone, catalog # SH30071-31) was added. This media was replaced with DMEM high glucose (Life Technologies, catalog #11965-092) containing 10% FBS 24 hours post-transfection. Forty eight hours after transfection, coverslips were fixed in 1% formaldehyde and permeabilised with 1% Triton X-100 where indicated in the figure. Visualization of Oreo was achieved using a mouse monoclonal antibody against the V5 tag (Invitrogen, catalog #46-0705 ) followed by a fluorescein conjugated secondary antibody (Pierce, catalog #31569) As a control for cell surface staining, some coverslips were treated with a mouse monoclonal antibody against EGF receptor (Oncogene Research Products, catalog # GR13), a well characterized cell surface protein. The fluorescent dye DAPI was used to visualize cell nuclei. Coverslips were viewed on a Leica DMLB inverted fluorescent microscope and images collected with a Cohu CCD camera using Leica QFISH version Y 2.3.0.

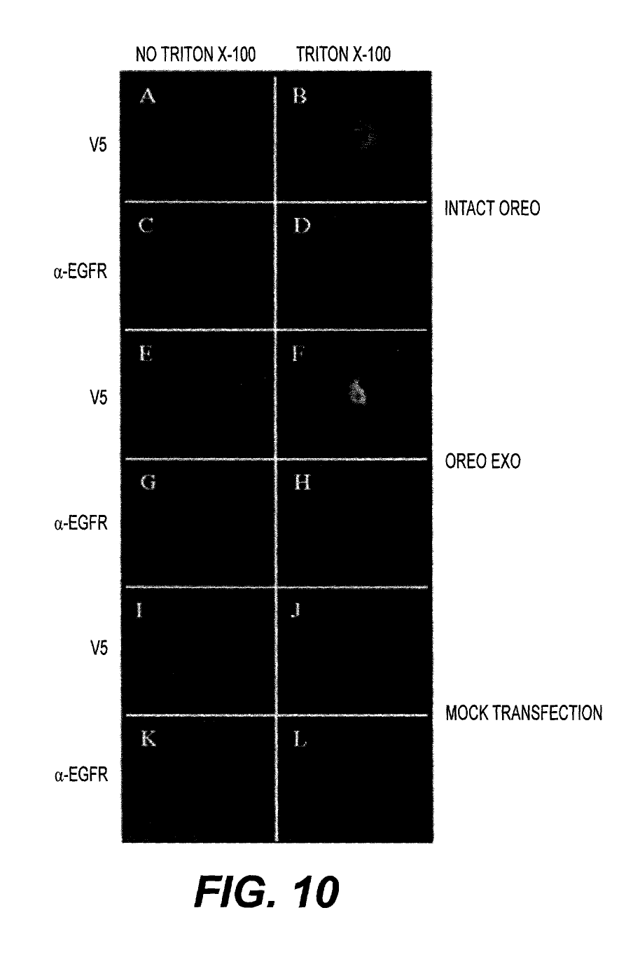

FIG. 10 depicts the results of the localization experiments. Panels A and B show localization of intact-Oreo in Triton permeablized and unpermeabilized cells. Oreo is found localized on the cell surface as demonstrated by staining in the absence of Triton (panel A). This staining pattern is confirmed as cell surface by comparison with the EGF receptor staining seen in panels C and D. In contrast, Oreo-exo is completely absent from the cell surface (panel E) and is only found intracellularly (panel F.) This result clearly demonstrates that the predicted transmembrane domain of Oreo (present in the intact Oreo construct only) is indeed a membrane anchor and acts as a signal to direct Oreo to the cell surface. This experiment confirms the prediction that Oreo is a Type II transmembrane protein. The remaining panels are controls showing EGF receptor cell surface staining (G-H) and the absence of Oreo staining in mock transfected cells (I-J).

### EXAMPLE 13

### Generation of Stable CHO Cell Lines Expressing Oreo

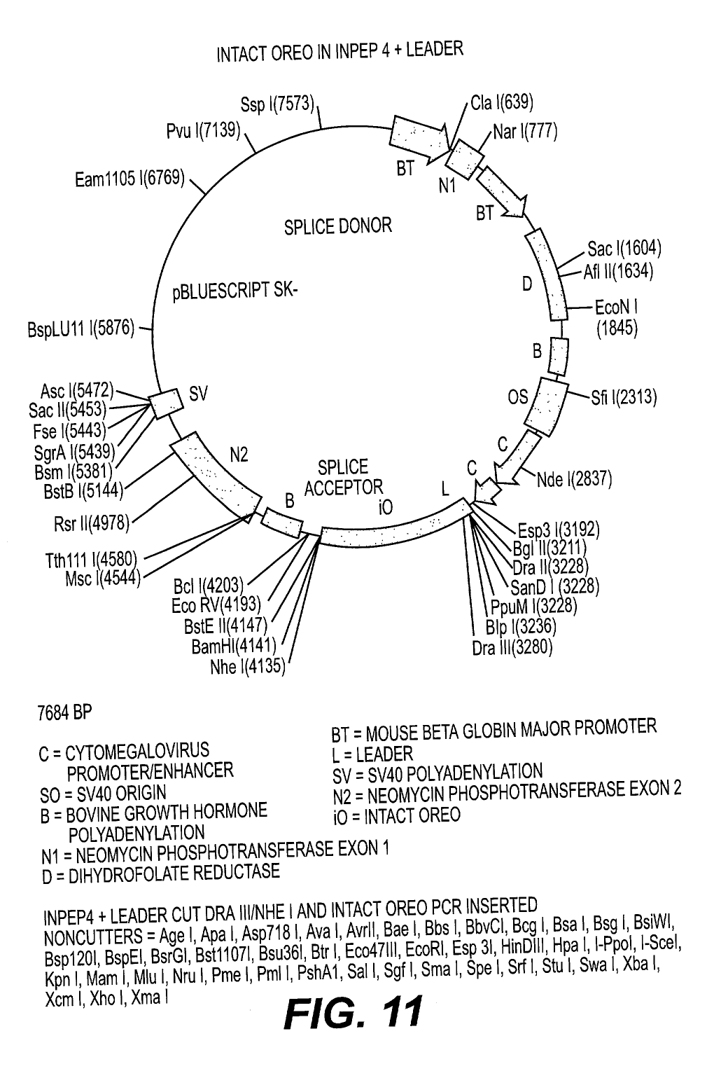

The intact Oreo and Oreo-exo constructs were inserted into the mammalian expression vector INPEP4+ Leader for expression in Chinese Hamster Ovary (CHO) cells. FIG. 11 depicts the plasmid maps for each of these vectors. The DNA elements comprising the INPEP4+ Leader vector are detailed in U.S. Pat. Nos. 5,648,267, 5,773,779, 6,017,773 and 6,159,730 by Reff et. al. Plasmid DNA for both Oreo constructs in INPEP4+Leader were prepared using the EndoFree Plasmid Maxi Kit (Qiagen corporation, catalog # 12362). Plasmid DNAs were linearized by overnight digestion with Pac I (NEB cat. #R0547L), then purified by phenol:CHCl₃ extraction and isopropanol precipitation. Linearized vectors were introduced into DHFR-CHO DG44 (Urlaub et al, 1986. Som,. Cell and Mol. Gen., 12:255-566) cells by electroporation as follows:

Exponentially growing cells were harvested by centrifugation and resuspended in sterile PBS at a concentration of 10⁷cells/mL. 0.4mL of cell suspension was mixed with 1-5 μg linear DNA and shocked using a BioRad Gene Pulser II (cat. #165-2105) and Capacitance Extender Plus (BioRad, cat. #165-2108) with instrument settings of 350V and 600□ F. Shocked cells were mixed with 20 mL growth media (CHO S-SFM II supplemented with HT, Life Technologies cat. #s31033-020, 11067-030) and plated in 96 well culture plates. Forty-eight hours after electroporation, plates were fed with G418 selection media (CHO S-SFM II media+HT+400 μg/mL G418, Life Technologies cat. #10131-035).

Plates were maintained in selection media for 30 days, during which time wells exhibiting cell growth were expanded into T150 flasks, and Oreo expression was measured by a relative quantitative RT-PCR (RQ RT-PCR) assay. Total RNA was isolated from each expanded culture using the RNeasy kit (QIAGEN, cat. #74104). cDNA was subsequently synthesized from the total RNA using Superscript II First-Strand Synthesis System for RT-PCR (Life Technologies cat. #11904-018). RQ RT-PCR assays were performed using Ambion's QuantumRNA Classic 18S Standards (cat. #1716.) An internal fragment of Oreo corresponding to nucleotides 469-769 was PCR generated in the assay and used as a measure of Oreo message. Cultures exhibiting the highest level of Oreo message were expanded into 125 mL spinner flasks for long term culture.

Oreo protein purified from these cultures may be purified and used for monoclonal antibody production by immunizing BALB/c mice. Details of the immunization methods to be used can be found in Kilpatrick et. al., 1997. Hybridoma, 16;381-389. High titer antibodies generated by this methodology will then be used for immunohistochemical analyses to further elucidate the tissue specific expression of this protein.

Plasmid DNA for both Oreo constructs in INPEP4+Leader was used to immunize BALB/c mice for the purpose of generating mouse polyclonal antisera against Oreo. The methodology for this DNA immunization is found in Chowdhury, Partha S. et. al. 1998. Isolation of a High-Affinity Stable Single-Chain Fv Specific for Mesothelin from DNA-immunized Mice by Phage Display and Construction of a Recombinant Immunotoxin with Anti-Tumor Activity. Proc. Natl. Acad. Sci. USA., 95, pp 669-674. Screening of these antisera awaits the purification of mammalian expressed Oreo. If high titer antibodies to Oreo are identified, they will be used for immunohistochemical analyses to further elucidate the tissue specific expression of this protein.

### EXAMPLE 14

### Generation of Stable CHO Cell Line Expressing a Tagged Version of Oreo

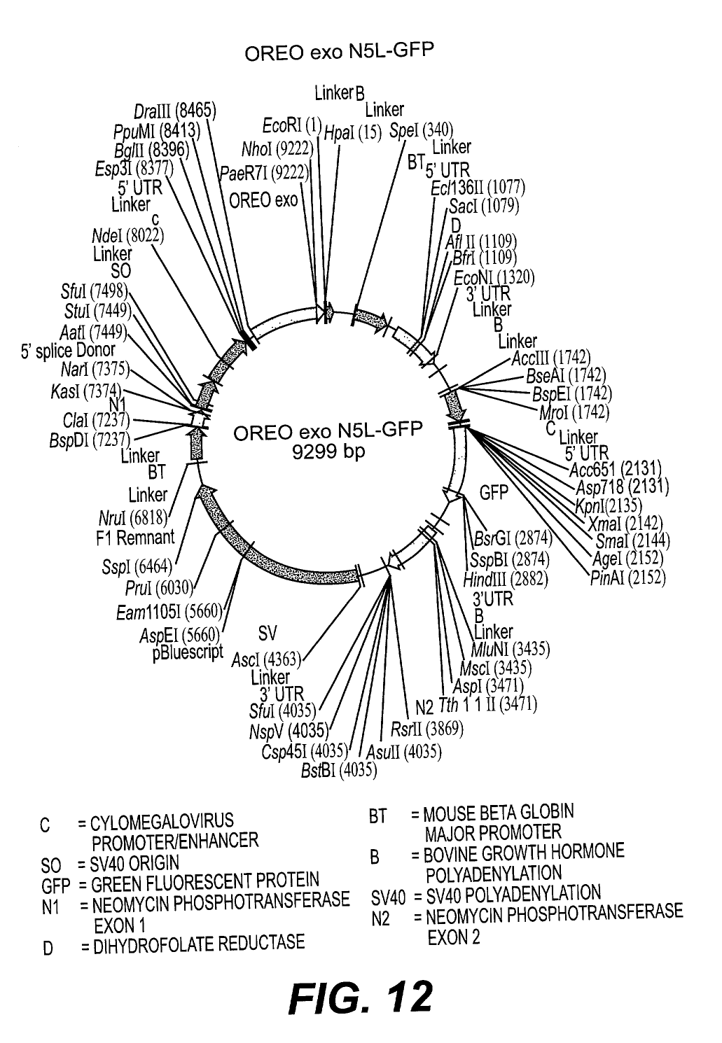

OREO-exo was inserted into the mammalian expression vector N5L-GFP in frame with an N-terminal leader sequence to target the protein for secretion. The C-terminal end of the construct contains a myc epitope tag and a 6×His tag for purification. Both of these tags were derived from the vector pSecTag2 (Invitrogen, catalog # V90020), details of which can be found in the Invitrogen catalog. The GFP component of the vector was derived from the mammalian expression vector pd2EGFP-1 (Clontech catalog #6008-1), details of which can be found in Clontech's Living Colors Users Manual (catalog # PT2040-1) The remaining DNA elements of the vector were derived from the same source as the INPEP4+Leader vector, details of which can be found in the patents referenced previously. FIG. 12 depicts the vector map of this construct.

Vector DNA was linearized by overnight digestion with the restriction enzyme Pci I (New England Biolabs, catalog # V0275S), then purified by ethanol precipitation. Linearized vector was introduced into DHFR-CHO DG44 cells essentially as described previously, with the following two modifications; 10 electroporations were performed, each using 40 μg DNA, and after shocking, cells were pooled, resuspended in 100 mL growth medium and cultured in a 125 mL spinner flask. Forty-eight hours after electroporation, cells were fed with G418 selection medium (described above) and maintained in this medium for 20 days. After this time, the culture was pelleted, washed with 1×PBS/10% FBS and resuspended in 1×PBS/10% FBS at a concentration of 5×10⁶ cells/mL. The culture was then sorted based on GFP fluorescence levels using a Becton Dickinson FACS Advantage SE machine and CellQuest Software at the UCSD Flow Cytometry Facility. Wild type DG44 cells were used as negative control to establish background autofluorescence. 6×10⁶ cells were sorted and two populations were collected based on GFP flourescence levels. The populations comprised 9.16% of the total cell population exhibiting bright flourescence, and 65.18% exhibiting dim flourescence. These cell populations were returned to routine culture in selection medium, thereby establishing stable GFP and Oreo expressing CHO cell lines.

Oreo protein purified from these cell lines may be purified and used for monoclonal antibody production by immunizing BALB/c mice. (Details of the immunization methods to be used can be found in Kilpatrick et. al., 1997. Hybridoma, 16;381-389) High titer antibodies generated by this methodology will then be used for immunohistochemical analyses to further elucidate the tissue specific expression of this protein.

While the invention has been described with respect to certain specific embodiments, it will be appreciated that many modifications and changes thereof may be made by those skilled in the art without departing from the spirit of the invention. It is intended, therefore, by the appended claims to cover all modifications and changes that fall within the true spirit and scope of the invention.

What is claimed is:

1. An isolated nucleic acid sequence that is expressed by human ovarian cancer cells selected from the group consisting of:
(i) the nucleic acid sequence contained in FIG. 1(a);
(ii) the nucleic acid sequence contained in FIG. 4(a) or 4(b), or a nucleic acid sequence containing both the sequences contained FIGS. 4(a) and 4(b);
(iii) the nucleic acid sequence in FIG. 7(a);
(iv) variants thereof, wherein such variants have a nucleic acid sequence that is at least 70% identical to the sequence of (i) or (ii) when aligned without allowing for gaps; and
(v) fragments of (i), (ii), or (iii) having a size of at least 20 nucleotides in length.

2. The nucleic acid sequence of claim 1 which comprises the nucleic acid sequence contained in FIG. (a).

3. The nucleic acid sequence of claim 1 which comprises the nucleic acid sequence contained in FIG. 4(a) or 4(b).

4. The nucleic acid sequence contained in FIG. 7(a).

5. A primer mixture that comprises primers that result in the specific amplification of one or both the cancer genes identified in claim 1.

6. A method of detecting ovarian cancer comprising (i) obtaining a human ovarian cell sample; and (ii) determining whether such cell sample expresses an ovarian cancer gene according to claim 1.

7. The method of claim 6, wherein said method comprises detecting the expression of said ovarian cancer gene using a nucleic acid sequence that specifically hybridizes thereto.

8. The method of claim 6, wherein said method comprises detecting the expression of said ovarian cancer gene using primers that result in the amplification thereof.

9. The method of claim 6, wherein the expression of said ovarian cancer gene is detected by assaying for the antigen encoded by said gene.

10. The method of claim 9, wherein said assay involves the use of a monoclonal antibody or fragment that specifically binds to said antigen.

11. The method of claim 10, wherein said assay comprises an ELISA or competitive binding assay.

12. An antigen expressed by human ovarian cancer cells that is selected from the group consisting of:
(i) the antigen encoded by the nucleic acid sequence in FIG. 1 (a);
(ii) the antigen encoded by the nucleic acid sequence in FIG. 4(a) and 4(b);
(iii) the antigen having the amino acid sequence in FIG. 7(b); and
(iv) fragments or variants thereof that bind to or elicit antibodies that specifically bind the antigen of (i) or (ii).

13. An ovarian antigen having the amino acid sequence in FIG. 7(b) or an antigen fragment thereof.

14. A monoclonal antibody or antigen-binding fragment thereof that specifically binds to an antigen according to claim 12.

15. A monoclonal antibody or fragment that specifically binds the antigen of claim 13.

16. The antigen of claim 12 or 13 which is attached directly or indirectly to a detectable label.

17. The antibody of claim 14 or 15 which is attached directly or indirectly to a detectable label.

18. A diagnostic kit for detection of ovarian cancer which comprises a DNA according to claim 1 and a detectable label.

19. A diagnostic kit for detection of ovarian cancer which comprises primers according to claim 2 and a diagnostically acceptable carrier.

20. A diagnostic kit for detection of ovarian cancer which comprises a monoclonal antibody according to claim 14 or 15 and a detectable label.

21. A method for treating ovarian cancer which comprises administering a therapeutically effective amount of a ribozyme or antisense oligonucleotide that inhibits the expression of a gene according to claim 1.

22. A method for treating ovarian cancer which comprises administering a nucleic acid sequence that specifically binds a gene according to claim 1, which is directly or indirectly attached to an effector moiety.

23. The method of claim 22, wherein said effector moiety is a therapeutic radiolabel, enzyme, cytotoxin, growth factor, or drug.

24. A method for treating ovarian cancer comprising administering a therapeutically effective amount of an antigen according to claim 12 or 13 and an adjuvant that elicits a humoral or cytotoxic T-lymphocyte response to said antigen.

25. A method for treating ovarian cancer comprising administering a therapeutically effective amount of a monoclonal antibody or fragment according to claim 14 or 15, optionally directly or indirectly attached to a therapeutic effector moiety.

26. The method of claim 25, wherein said effector moiety is a radiolabel, enzyme, cytotoxin, growth factor, or drug.

27. The method of claim 26 wherein the radiolabel is yttrium.

28. The method of claim 26 wherein the radiolabel is indium.
